(19) United States
(12) Patent Application Publication
(10) Pub. No.: US 2014/0148823 A1
(43) Pub. Date: May 29, 2014

(54) TREATMENT OF NAIL DISEASE

(71) Applicant: Thomas Patrick Fitzsimons, Swarthmore, PA (US)

(72) Inventor: Thomas Patrick Fitzsimons, Swarthmore, PA (US)

(73) Assignee: KERATHIN LLC, Chester, PA (US)

(21) Appl. No.: 13/885,527

(22) PCT Filed: Nov. 14, 2011
(86) PCT No.: PCT/US2011/060591

§ 371 Date: Aug. 2, 2013

Related U.S. Application Data
(60) Provisional application No. 61/415,527, filed on Nov. 19, 2010.

(51) Int. Cl. A61B 17/54

(52) U.S. Cl. CPC A61B 17/54
USPC 606/131

(57) ABSTRACT

A method and corresponding device and kit for treating a human nail may include applying to a surface of said nail distal to the bed of the nail an abrasive surface oscillating through a length in a plane substantially parallel to said nail surface at an area of application and urging the abrasive surface against the area of application with a force substantially normal to the nail surface at the area of application, wherein the urging force taken together with the extent of oscillation of the abrasive surface at the area of application is sufficient to abrade the material of the nail from said surface, but is substantially incapable of damaging living tissues from areas surrounding the nail.

### CROSS-REFERENCE TO RELATED APPLICATION

This application claims the benefit of U.S. Patent Application Ser. No. 61/415,527 filed on Nov. 19, 2010, the disclosure of which is hereby incorporated by reference as if set forth in its entirety herein.

### BACKGROUND

Fungal infections of the nail are a persistent problem for tens of millions of people. These infections start at the free end of the nail and often invade the nail frequently leading to nail dystrophy or abnormal nail anatomy such as thickening and separation of the nail from the nail bed. The nail is relatively impermeable to antifungal medications. Therefore, effective treatment may involve medications taken orally or applied topically for a period of many months. These infections are especially prevalent in diabetics.

Recent studies have shown enhancement of these medications in the presence of aggressive debridement of the nail. (Malay, D. et. al; Efficacy of Debridement Alone Versus Debridement Combined with Topical Antifungal Nail Lacquer for the Treatment of Pedal Onychomycosis: A Randomized, Controlled Trial; Journal of Foot and Ankle Surgery; Volume 48, Issue 3, May 2009, Pages 294-308). This debridement is accomplished using clippers, scissors, manual files or nail grinders. Nail grinders are machines consisting of a motor connected to a shaft which rotates. On the shaft is a burr, rasp or grinding stone. See for example the Sprint 100 (http://www.algeos.com/acatalog/Sprint_{—}100.html). This burr is applied to the nail to quickly abrade portions of the nail away. This reduces fungal burden in the nail and enhances the effectiveness of medication by allowing medication easier access to infected nail.

While nail grinders have a beneficial effect they also have risks. Typically these grinders rotate in the thousands to tens of thousands of revolutions per minute. Consequently the abraded portions of the nail are sprayed into the environment. This can lead to allergic reactions and asthma in patients and healthcare providers. Studies have demonstrated a high rate of sensitivity to common fungal organisms infecting the nail among podiatrists. (Davies, R.; Human nail dust and precipitating antibodies to Trichophyton rubrum in chiropodists; Clinical & Experimental Allergy; Volume 13, Issue 4). Literature also shows increased sensitivity to common nail pathogens among chiropodists and podiatrists caused by airborne nail particles resulting from grinding (G. Ward; Trichophyton Asthma Sensitisation of Bronchi and Upper Airways to Dermatophyte Antigen; The Lancet, Volume 333, Issue 8643, Pages 859-862). In addition, the aerosolized fungus is known to cause opportunistic infections in immune-compromised patients and diabetics. (Gupta A, et al; The use of terbinafine in the treatment of onychomycosis in adults and special populations: a review of the evidence; Journal of Drugs in Dermatology, May-June, 2005; Winston, J, et al; Treatment of Onychomycosis in Diabetic Patients; Clinical Diabetes October 2006 vol. 24 no. 4 160-166).

Nail grinders may have a built in vacuum system to control the aerosolization of infected nail particles. U.S. Pat. No. 3,126,021 is an example of this approach which demonstrates the use of an air intakes system to control dust. An example of a commercially available nail grinder for use in medical offices and nail care spas (from http://www.algeos.com/acatalog/Sprint_Zero.html, accessed Sep. 13, 2010) includes a grinder with a handle which contains an integral dust collector system which leads to a collection bag within the case to minimize environmental nail dust.

In addition, the high speed rotary motion of these nail grinders can be shown to damage skin. This may limit their safe use to the central portion of the nail, away from the skin tissues surrounding the nail. While fungal infection can affect all portions of the nail, including the portion at the nail fold and at the free end of the nail, use of grinders in these areas risks skin damage.

### SUMMARY

A method for treating a human nail may comprise applying to a surface of said nail distal to the bed of the nail an abrasive surface oscillating through a length in a plane substantially parallel to said nail surface at an area of application and urging the abrasive surface against the area of application with a force substantially normal to the nail surface at the area of application, wherein the urging force taken together with the extent of oscillation of the abrasive surface at the area of application is sufficient to abrade the material of the nail from said surface, but is substantially incapable of damaging living tissues from areas surrounding the nail.

A device for treating a human nail may include a handle, a surface for carrying an abrasive, an actuator configured to vibrate or oscillate said abrasive surface, and a stall feature configured to slow or stall the actuator when said abrasive surface is applied with a force substantially normal to an area of application on a surface of said nail distal to the nail bed that is greater than the normal force required, together with a length of oscillation of the abrasive surface applied to the area of application, to cause abrasion of a keratin matrix of the nail without damaging the living tissues surrounding said nail. The device may further include a control system with a sensor configured to sense the normal force of the abrasive surface against the area of application and to activate the stall feature if the normal force is great enough to damage the living tissues surrounding the nail.

A kit for the treatment of human nail may include a device for treating the human nail comprising a handle, an actuator configured to oscillate an abrasive surface, and a stall feature configured to stall the actuator when the abrasive surface is applied with a force substantially normal to an area of application on a surface of said nail distal to the nail bed that is greater than the normal force required, together with a length of oscillation of the abrasive surface applied to the area of application, to cause abrasion of a keratin matrix of the nail without damaging the living tissues surrounding said nail. The kit may further include a plurality of disposable disks, each having an abrasive surface, wherein each disposable disk is configured to attach to a device for treating a human nail.

### BRIEF DESCRIPTION OF THE DRAWINGS

FIG. 1 illustrates the anatomy of a human nail and surrounding tissue;

FIG. 2 shows a magnified perspective view schematic of Section A of the nail shown in FIG. 1;

FIG. 3 shows a schematic of one embodiment of a device for treating mammalian nails as shown and described herein; and

FIG. 4 shows the grinder shown in FIG. 3 and a plurality of disposable disks;

FIG. 5 shows a cross-sectional schematic of the grinder shown in FIGS. 3 and 4; and

FIG. 6 shows a top plan schematic view of the abrasive surface shown in FIGS. 3, 4, and 5.

### DETAILED DESCRIPTION OF ILLUSTRATIVE EMBODIMENTS

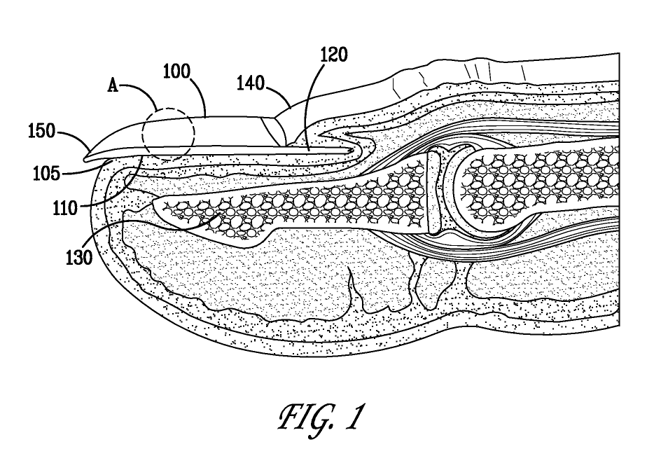

FIG. 1 shows the anatomy of a human nail 100 and the surrounding living skin tissue 140. The rigid nail 100 grows along a nail bed 110 distally from the nail root 120. The phalanx 130 extends underneath the nail bed 110 surrounded by living skin tissue 140. The hyponychium 105, also known as the quick, is the epithelium located beneath the nail 100 at the junction between the free end 150 of the nail and the skin tissue 140 of the fingertip. It forms a seal that protects the nail bed 110. The free end 150 of the nail 100 extends distally beyond the nail bed 110.

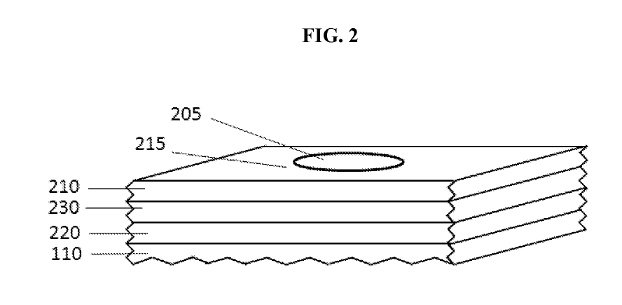

Section A of FIG. 1 is shown as a magnified schematic in FIG. 2. As shown, the human nail 100 sits on the nail bed 110 and is multilayered. The horny layer 210 and the bottom layer 220 of the nail 100 have generally longitudinally oriented fibers. The middle layer 230 consists of a matrix of generally orthogonally oriented fibers. The differing orientations of the fibers of the horny layer 210, bottom layer 220, and middle layer 230 provide both hardness for support and flexibility to prevent tears and fractures. Onychomycotic infection may invade the nail 100, entering through the hyponychium 105 and spreading through to the middle layer 230. Such an infection can lead to nail thickening and disfigurement. Because the nail 100 is relatively hard, treatment for infection of the nail 100 may not be able to penetrate to the middle layer 230 where the infection often spreads.

While the nail 100 is relatively hard and brittle, the surrounding living skin tissue 140 is relatively soft and elastic. Due to these properties, when stress is applied in certain directions, the nail 100 has a relatively lower elastic limit than skin tissue. Accordingly, at least for force being applied substantially along a plane of an area of application on the skin and nail, generally higher levels of stress may be applied to the skin tissue 140 as compared to the nail 100 without causing permanent deformation or damage of the skin tissue 140 such as tearing, abrasion, or bruising. In other words, a stress level that may cause abrasion of the nail 100 is not necessarily high enough to cause any damage to the skin tissue 140 due to the relative difference in the elastic limits of the nail 100 and the skin tissue 140.

Also, the Young's Moduli of both the skin tissue 140 and nail 100 are vastly different and vary with age and hydration. In scientific literature, the Young's modulus of the skin varies between 0.42 MPa and 0.85 MPa for torsion tests (Agache P G, Monneur C, Leveque J L, de Rigal J. Mechanical properties and Young's modulus of human skin in vivo. Arch Dermatol Res 1980; 269:127-33). Pailler-Mattei C, et. al. report that an average value of the skin Young's modulus between 4.5 kPa and 8 kPa for indentation techniques (C. Pailler-Mattei, S. Bec, H. Zahouani. In vivo measurements of the elastic mechanical properties of human skin by indentation tests. Medical Engineering & Physics 30 (2008) 599-606). Baden reports the Young's Modulus of the nail to be between 4.3±0.3·GPa transversely, and 2.1±0.3·GPa longitudinally (Baden, H. P. (1970). The physical properties of nail. J. Invest. Dermatol. 55, 115-122.). The tissue underlying the nail is a complex mixture of subdermal tissues including fat, bone and dermal layers. Human skin is a living complex material, composed of several heterogeneous layers. It is mainly composed of three layers: epidermis, dermis, and hypodermis which is an extremely viscous and soft layer. The dermis consists of a network of collagen with interspersed elastic fibers, and lymphatic elements, all covered by an epidermal layer of partially keratinized cells that are progressively dehydrated during their migration to the outer surface. The thickness of each skin layer varies as a function of age, body zone or hydration (Agache P. Physiologie de la peauet exploitations fonctionnellescutan'ees. Paris: Editions M'edicalesInternationales; 2000, C. Pailler-Mattei).

Indentation techniques have been used to measure the Young's modulus of both nail and skin and demonstrate a difference in Young's Modulus of several orders of magnitude. Agache, et. al. have shown that the viscoelastic tissue of the skin can easily accept rotary motion over an arc of 6 degrees without and damage to the skin structure. (P. G. Agache, C. Monneur, J. L. Leveque, and J. De Rigal; Mechanical Properties and Young's Modulus of Human Skin in Vivo; Arch Dermatol Res 269, 221-232 (1980)). Similarly, Faran, et al have demonstrated that exfoliation of the distal portion of the nail can occur with forces of 0.01 kN over a distance as short as 1.0 mm. (L. Farran, A. R. Ennos, M. Starkie, S. J. Eichhorn; Tensile and shear properties of fingernails as a function of a changing humidity environment; Journal of Biomechanics 42 (2009) 1230-1235).

Despite these measurement differences the Young's Modulus of skin and the nail vary by orders of magnitude. Given the highly viscoelastic nature of the skin tissue 140 and the relatively rigid and inelastic properties of the nail 100, it is possible to use the differing strain response to stress of the skin tissue 140 and the nail 100 to design an article or device for treating a human nail. Specifically, such a device may have an abrasive surface such as a disk or pad capable of delivering sufficient motion, when combined with a normal force against the nail to create a stress that is above the elastic limit of the nail 100 so that the device will shear portions of the nail 100 away. This stress may also be below the elastic limit of the skin tissue 140, thereby harmlessly stretching the skin tissue 140. In other words, such a device may utilize the relative difference between elastic limit of the nail 100 and the surrounding tissue 140 to safely and effectively treat Onychomycosis and other nail dystrophy.

Tissue surrounding the nail may be damaged by nail grinders in a number of ways. They may be bruised by application of too much force normal to the tissue, they may be torn by overstretching the elastic limit of the tissue or they may be burned by the friction generated by the local application of force in one place for a period of time that goes beyond the ability of the surrounding tissue to dissipate heat. Heat of friction is a function of the normal force and the oscillatory speed among other things. The stress level delivered by such a device is a function of the motion of the abrasive surface against the nail 100. As described in more detail below, this motion may be characterized by a length, such as a maximum arc length of oscillation of the edge of the abrasive surface as it contacts the nail 100. Arc length is a function of the degree of oscillation and the radius of the portion of the abrasive surface that contacts the nail. It will also be appreciated that the oscillation may be linear rather than rotational and that the length can be the maximum linear displacement of the abrasive surface.

To the extent that normal force against the nail must be sufficient to prevent slippage, the normal force also effects the stress level delivered by such a device. The normal force may be controlled by using an actuator with a stall feature that slows or stops actuation when the normal force applied to the nail 100 exceeds a certain level. This will alert the user or prevent damage from either bruising or the heat of friction. Experiments have shown that motors with a stall torque of approximately 130 gm*cm effectively remove a portion of the nail 100 without damaging the surrounding skin tissue 140. For these experiments, the arc length ranges from 0.20 inches-0.23 inches (4.5 mm-5.9 mm).

A device as described above may be used to provide methods for treating the nails of mammals, especially human beings, which are infected with fungus or have other diseases or conditions causing dystrophy of the nails. In accordance with certain embodiments, improved chemotherapeutic or other treatment of such nails is accorded by the selective and relatively non-infective debridement of portions of the nails prior to application of the agent or other treatment modalities. An oscillating abrasive pad is urged against the nail to cause debridement and/or to debulk the nail. When compared to the axially rotating motion of other devices such as a Dremel tool, this oscillating motion minimizes the generation of actually or potentially infectious, fugitive dust.

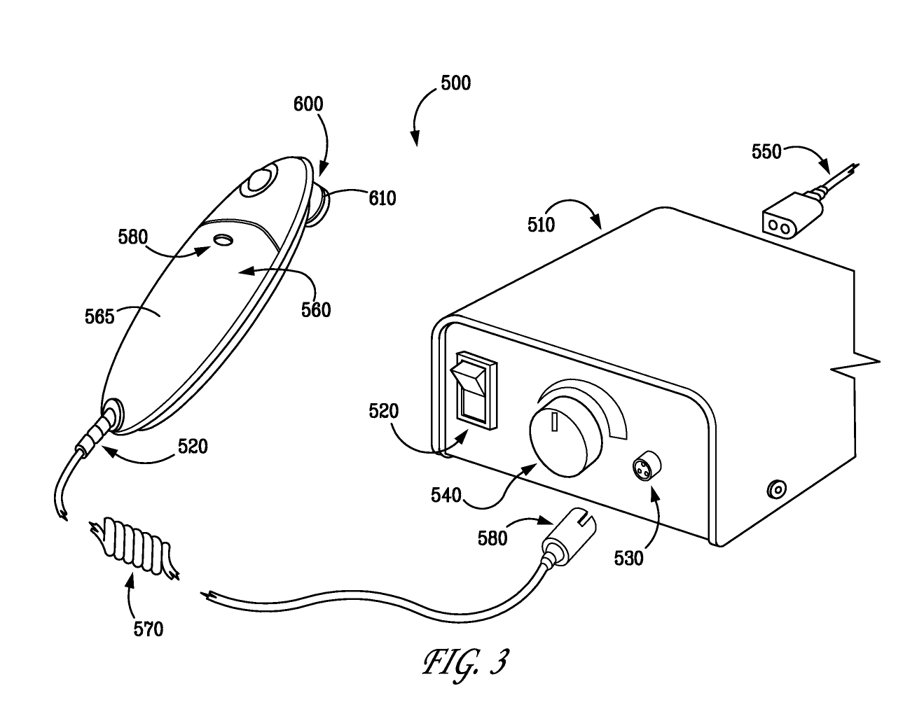

One embodiment for a device 500 for treating nails in mammals is shown in FIGS. 3-6. A controller 510 contains an on/off switch 520 a connecting jack 530, a dial 540 and a power plug 550 for connection to a main power supply (not shown). The controller 510 is connected to a grinder 560 by a power conduit 570. The grinder 560 has a local on/off switch 580 for remotely controlling the power delivery from the controller 510. Contained within the grinder 560 may be a vessel or compartment (not shown) to hold and deliver chemotherapeutic agent, liquids, emoluments or other desired substances unto the nail. In another embodiment, the device 500 may have a co-registered vacuum system (not shown) to further control dust in the environment. As will be appreciated, other design implementations could be used such as rechargeable grinders without power cords and with variable speed control built into the grinder handle.

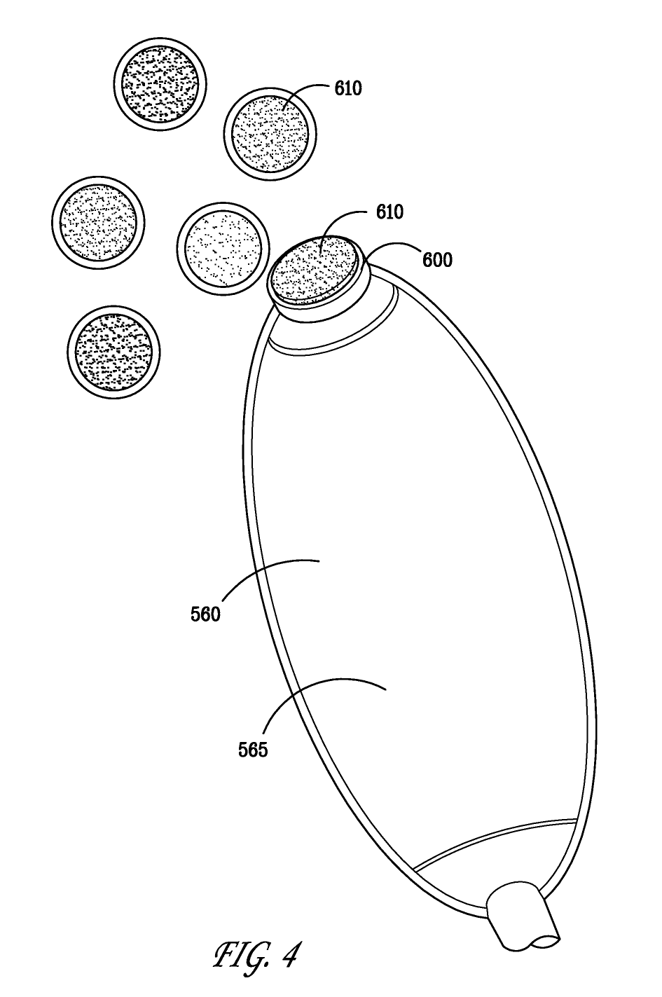

As further shown in FIG. 4, the grinder 560 has a handle 565 and an abrading portion 600. The abrading portion 600 may be a pad or disk that has a surface 610 for carrying an abrasive. In one embodiment, the surface 610 includes emery paper with a 130 grit. In an example commercial embodiment, several abrading portions 600 in the form of disposable disks may be sold. These disposable disks may have abrasive portions with the same or different grit ratings. For example, the device 500 may be sold with 3 disposable disks. One disk may have a grit rating of 60; another disk may have a grit rating of 80; a third disk may have a grit rating of 120.

In some embodiments, the surface 610 for carrying an abrasive may have a grit rating in the range of 50 to 4000 depending on the particular need of the patient or user. The grit material of the abrasive surface 610 may be aluminum oxide, silicon carbide or any other material known in the art. Further, in some embodiments, the surface 610 may be used with a wetting solution such as water or alcohol or a grinding solution or compound such as 3M™ Finesse-it™ Compounding Material. This material may also be a chemotherapeutic agent, such as terbinafine hydrochloride, urea or undecylenic acid as a solid or solution. The surface 610 may have a conformable backing to keep the force applied to the nail normal to the nail along the curved surface of the nail. The conformable backing may include foam that provides flexibility to the abrasive surface so that the surface can curve around the curved nail. It may be waterproof for use in conjunction with topical anti-fungal medications or with liquids designed to further suppress nail detritus from moving into the surrounding environment.

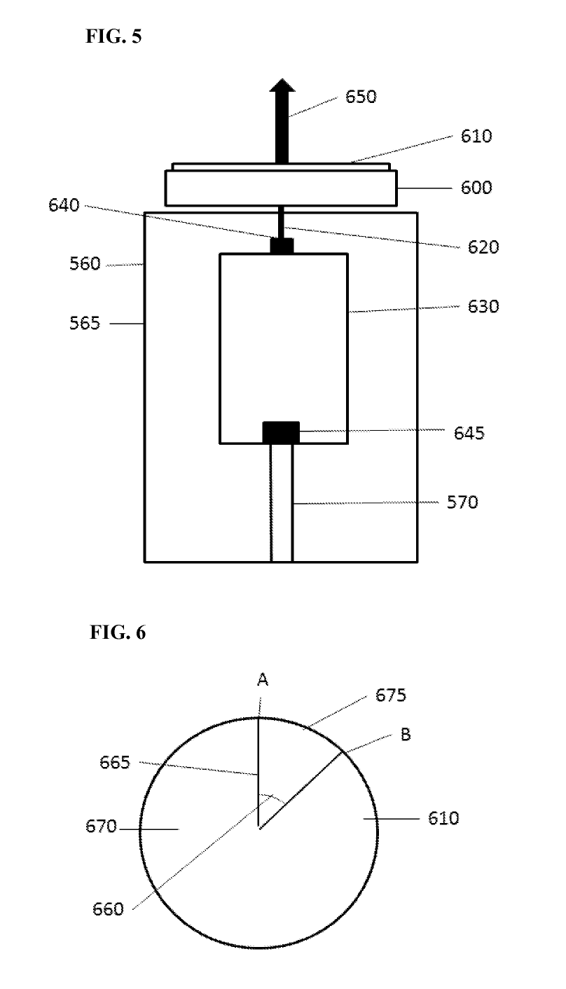

As shown in the schematic of the grinder 560 in FIG. 5, the handle 565 may incase an actuator or motor 630 that drives an output 620 which is configured to oscillate the abrasive portion 600. The motor 630 may directly connect to the abrading portion 600 or the motor 630 may be connected to the abrading portion 600 by a rigid or flexible drive shaft or other drive train. The actuator or motor 630 may be driven by the power conduit 570 which connects to the controller 510 shown in FIG. 5. Normal force 650 is the normal force delivered to the nail 100. Force 650 may be measured by sensor 640. The controller 510 may be configured to activate a stall feature 645 that slows or stops oscillation of the actuator 630 when the normal force 650 measured by the sensor 640 exceeds a certain level. In this way, the controller 510 may function to stop oscillation if the normal force is great enough to cause damage to the living skin tissue 140 surrounding the nail 100. The maximum level of normal force may be set by the dial 540. Alternatively, the device may have a sacrificial link or drive train element designed to break is a certain force rating is surpassed

As shown in FIG. 4, the handle portion 565 that may be fashioned for the careful application of oscillatory, abrasive force upon the nail 100. The handle 565 may further allow for control of the urging force as well as exact placement upon the nail 100 so that the nail may be debrided while avoiding damage to surrounding skin tissue 140. This may be accomplished by using an oscillating motion that is constrained by its design to apply stress above the level needed to abrade the nail 100 by overcoming the elasticity of the nail, but below the level needed to overcome the elasticity of the surrounding skin 140.

By way of example, the actuator 630 may be a rotary brushed motor such as Maxon RE 16, Portescap16GE88, Faulhaber 2232-006SR. Any actuator with similar properties may be used. The actuator 630 may drive abrading portion 600 in an oscillating motion along a length 675 that may be an arc length. It will also be appreciated that the actuator 630 may drive the abrading portion 600 in a linearly reciprocating fashion through a length 675. In the exemplary embodiment shown in FIG. 6, this oscillating motion may be defined by an angle of oscillation 660. The angle of oscillation 660 together with the radius 665 of the abrading surface 610 determines length 675, defined as the distance between points A and B which is an arc length. Length 675 lies in a plane 670 which is defined by the abrading surface 610. During use of the device 600, plane 670 is substantially parallel to the surface 215 of the nail 100 at an area of application 205 (as seen in FIG. 2). As indicated above, the stress applied to the nail 100 and skin 140 is a function of the normal force 650 and the length 675. The exemplary embodiment of the device uses the relative difference in the elastic limits of the nail 100 and the skin 140 to provide a method to safely debride the nail, especially near the nail fold or close to the nail bed. This may debulk an infected nail and reduce fungal load to improve the effectiveness of chemotherapeutic agents against nail fungus. In addition, this oscillating motion prevents the centrifugal force of the abrading portion of the device from spraying abraded portions of infected nail into the environment because the oscillating motion is in a single plane along a limited angle of rotation.

In one embodiment, the abrasive surface 610 may oscillate at a frequency between 100 and 30,000 oscillations per minute. In another embodiment, the abrasive surface 610 may oscillate at a frequency between 2,000 and 16,000 oscillations per minute. In yet another embodiment, the abrasive surface may oscillate at a frequency between 4,000 and 12,000 oscillations per minute.

In one embodiment, the angle of oscillation 660 may be between 2 and 45 degrees in the plane of the abrading surface. In another embodiment, the angle of oscillation 660 may be between 5 and 15 degrees in either direction from resting dead center. In yet another embodiment, the angle of oscillation 660 may range from 30 to 40 degrees. The abrading surface 610 may be any shape having a substantially planar abrasive surface in the plane of the oscillation.

The abrading surface may be urged against the nail 100 with the normal force 650. In one embodiment, this force is a force that is customary for the heavy filing of toenails. In one embodiment, the device 500 includes a sensor 640 that measures normal force 650. When normal force 650 is above a certain threshold, motor 630 may be programmed to stall. This stall force may be about 3 Mpa which is the force generally acknowledged sufficient to bruise skin (Bush M, et al The Response of Skin to Applied Stress: Investigation of Bitemark Distortion in a Cadaver Model; Journal of Forensic Sciences, January 2010, 55:1, 71-76). In another embodiment, the stall force may be in the range of 1-1.5 Mpa normal to the nail surface. In yet another embodiment, the stall force may be in the range of 1.5 to 2.5 Mpa.

In some embodiments, the power and speed will be higher than in other embodiments. For example, the stall force in one embodiment may be from 0.25-0.75 Mpa normal to the nail in order to maximize safety to surrounding skin. Such an embodiment may be used for diabetic patients. Embodiments with higher power and speed may be used in professional podiatrists' offices. Embodiments with lower power and speed may be used at home by patients for interim home care.

In accordance with some embodiments, the actuator 600 may have an adjustable or fixed speed and may be driven by an external power supply or an internal variable resistor or other speed control device. Power may be provided by an AC source optionally via a transformer. Alternatively articles in accordance with this invention may be powered by a self-contained battery, either rechargeable or replaceable.

In one embodiment, the abrasive surface 610 may have a grit rating between 5 and 1,500. In another embodiment, the grit rating may range from 100 to 300. In yet another embodiment, two or more disks may be used that have incremental grit values. In one embodiment, the abrasive surface 610 may be a reusable disk. In another embodiment, the abrasive surface 610 may be a disposable, single use disk which is not cleaned or re-sterilized.

### Example 1

A foot was placed on an “L” shaped stand consisting of a flat surface with 12 inch barrier at the edge of and orthogonal to the flat surface four inches removed from the long edge of foot, running parallel to the long axis of the foot and extending four inches fore and aft of the approximate center of the big toenail. The barrier's perpendicular surface and the four inches between the foot and the barrier were covered with double-faced tape. A circularly rotating abrading tool (Dremel model 395, grinding bit 952) was brought into contact with the toenail and allowed to run for 20 seconds. The double faced tape was then removed. This procedure was redone using a prototype of the present reciprocating abrading device. The two double-faced tape collection barriers were then examined with a magnifying glass. The results showed a slightly higher amount of toenail detritus on the flat surface with the circular abrading tool, but a markedly greater amount on the orthogonal surface. This is to be expected from the centrifugal force of the circular abrading means and verifies that the present reciprocal abrading means causes less environmental contamination during use as a result of the distinct difference in the motion of the abrading or grinding bit.

### Example 2

An electric motor was connected to a reciprocating gear driven disk. A 120 grit commercial sandpaper disc was attached. The oscillatory frequency of the disk was 800 oscillations per minute. The device was started and placed in contact with the nail. After five (5) seconds the device was removed and the area of the nail was examined using a magnifying glass. Obvious signs of nail abrasion were visible. Subsequently the same device was placed against both normal skin and the cuticle surrounding the nail and operated for a period of seconds. Magnified examination of both areas showed no damage swelling or redness to indicate any tissue damage. This experiment has been repeated with other grain sized commercial sandpaper, at varying speeds in excess of 8,800 oscillations per minute, for periods up to two (2) minutes and with reciprocating and vibratory motions. This has been repeated on dry nail as well as on nail wetted with an antifungal solution. All have achieved the same result, abrasion of the nail without damage to the surrounding dermal tissues.

Although the disclosure has been described in detail, it should be understood that various changes, substitutions, and alterations can be made herein without departing from the spirit and scope of the disclosure as defined by the appended claims. Moreover, the scope of the present disclosure is not intended to be limited to the particular embodiments described in the specification. As one of ordinary skill in the art will readily appreciate from the disclosure above, processes, machines, manufacture, composition of matter, means, methods, or steps, presently existing or later to be developed that perform substantially the same function or achieve substantially the same result as the corresponding embodiments described herein may be utilized according to the present disclosure.

What is claimed:

1. A method for treating a human nail comprising:
applying to a surface of said nail distal to the bed of the nail an abrasive surface oscillating through a length in a plane substantially parallel to said nail surface at an area of application; and
urging the abrasive surface against the area of application with a force substantially normal to the nail surface at the area of application, wherein the urging force taken together with the extent of oscillation of the abrasive surface at the area of application is sufficient to abrade the material of the nail from said surface, but is substantially incapable of damaging living tissues from areas surrounding the nail.

2. The method of claim 1 wherein the oscillating abrasive surface is operated under the control of a controller.

3. The method of claim 2 wherein the controller is configured to slow or stall the oscillation of the abrasive surface under conditions where the urging force taken together with the speed and extent of oscillation of the abrasive surface at the area of application is capable of damaging living tissue from areas surrounding the nail.

4. The method of claim 3 wherein the controller is configured to stall the oscillation when the urging force is equal to a preselected value.

5. The method of claim 3 wherein the controller is configured so that a preselected value may be selected from a set of values and the controller is further configured to stall the oscillation when the urging force is equal to the preselected value

6. The method of claim 1 wherein damaging living tissue comprises bruising, ripping, or burning.

7. The method of claim 1 wherein the abrasive surface has a grit rating of 5 to 1,500.

8. The method of claim 7 wherein the grit rating is 100 to 300.

9. The method of claim 1 wherein said abrasive surface oscillates at a frequency of 100 to 30,000 oscillations/minute.

10. The method of claim 9 wherein the frequency is 4,000 to 12,000 oscillations/minute.

11. The method of claim 1 wherein the urging force is 0.25 to 0.75 Mpa.

12. The method of claim 1 wherein the urging force is 1.0 to 1.5 Mpa.

13. The method of claim 1 wherein the urging force is 1.5 to 2.5 Mpa.

14. A device for treating a human nail comprising a handle, a surface for carrying an abrasive, an actuator configured to oscillate said abrasive surface, and a stall feature configured to slow or stall the actuator when said abrasive surface is applied with a force substantially normal to an area of application on a surface of said nail distal to the nail bed that is greater than the normal force required, together with a length of oscillation of the abrasive surface applied to the area of application, to cause abrasion of a keratin matrix of the nail without damaging the living tissues surrounding said nail.

15. The device of claim 14 further comprising a control system including a sensor configured to sense the normal force of the abrasive surface against the area of application and to activate the stall feature if the normal force is great enough to damage the living tissues surrounding the nail.

16. The device of claim 14 wherein said abrasive surface has a grit rating is 5 to 1500.

17. The device of claim 16 wherein the grit rating is 100 to 300.

18. The device of claim 14 wherein said abrasive surface oscillates at a frequency of 100 to 30,000 oscillations/minute.

19. The device of claim 18 wherein the frequency is 4,000 to 12,000 oscillations/minute.

20. The device of claim 14 wherein the required normal force is 0.25 to 0.75 Mpa.

21. The device of claim 14 wherein the required normal force is 1.0 to 1.5 Mpa.

22. The device of claim 14 wherein the required normal force is 1.5 to 2.5 Mpa.

23. A kit for the treatment of human nail comprising:
a) a device for treating the human nail comprising a handle, an actuator configured to oscillate an abrasive surface, and a stall feature configured to slow or stall the actuator when the abrasive surface is applied with a force substantially normal to an area of application on a surface of said nail distal to the nail bed that is greater than the normal force required, together with a length of oscillation of the abrasive surface applied to the area of application, to cause abrasion of a keratin matrix of the nail without damaging the living tissues surrounding said nail; and
b) a plurality of disposable disks, each having an abrasive surface, wherein each disposable disk is configured to attach to a device for treating a human nail.

24. The kit of claim 23 wherein the device further comprises a control system including a sensor configured to sense the normal force of the abrasive surface against the area of application and to activate the stall feature if the normal force is great enough to damage the living tissues surrounding the nail.

25. The kit of claim 23 wherein the plurality of disposable disks comprises at least three disposable disks.

26. The kit of claim 25 wherein the plurality of disposable disks comprises at least two disposable disks having abrasive surfaces with different grit ratings.

27. The kit of claim 23 wherein at least one of the abrasive surfaces has a grit rating of 5 to 1500.

28. The kit of claim 27 wherein the grit rating is 100 to 300.

29. The kit of claim 23 wherein at least some of the plurality of disposable disks have different grit ratings.

30. The kit of claim 29 wherein the plurality of disposable disks comprises at least one disposable disk with a grit rating of 60, at least one disposable disk with a grit rating of 80, and at least one disposable disk with a grit rating of 120.
